(19) United States
(12) Patent Application Publication
(10) Pub. No.: US 2020/0297449 A1
(43) Pub. Date: Sep. 24, 2020

(54) COVER FOR AN ULTRASOUND PROBE

(71) Applicant: SPT VILECON A/S, Hedehusene (DK)

(72) Inventor: Bjarne Andersen, Frederikssund (DK)

(73) Assignee: SPT VILECON A/S, Hedehusene (DK)

(21) Appl. No.: 16/651,613

(22) PCT Filed: Sep. 25, 2018
(86) PCT No.: PCT/EP2018/075867

§ 371 Date: Mar. 27, 2020

(30) Foreign Application Priority Data
Sep. 28, 2017 (EP) 17193616.4

(51) Int. Cl. A61B 46/10; A61B 42/10; B29C 41/14; B29C 41/00; A61B 46/00

(52) U.S. Cl. CPC A61B 46/10; A61B 42/10; B29K 2075/00; B29C 41/003; A61B 46/40; B29C 41/14

(57) ABSTRACT

The invention regards a flexible cover for an ultrasound device, the cover comprising polyurethane, wherein the cover is shaped as a sheath having a closed end, said shape is made by dip molding, and wherein the ultrasound attenuation is at least below 60% of the attenuation of latex, more preferably below at least 55%, 50%, 45%, 40%, 35%, or 30% below the attenuation of latex.

### FIELD OF INVENTION

The present invention relates to a cover for an ultrasound probe.

### BACKGROUND OF INVENTION

Medical devices used for patient examination and/or treatment is often protected by a cover. The cover may be disposable and/or easily sterilized, and thus expensive cleaning of the device as well as risks of spreading contagious diseases and cross-contamination between patients are reduced.

Covers for medical devices are typically made of elastomeric materials due to their mechanical properties. A cover must have sufficient tensile- and shear-strength as well as resiliency. Strength is required as the cover may be expanded during mounting and use, e.g. such that it can be formfitted or close fitted to the medical device to minimize air bubbles trapped inside the cover. Trapped air bubbles are for example unwanted between covers and probes for ultrasound devices, since air is a poor ultrasound conductor.

Resiliency, i.e. the ability to return to the original shape or configuration, is required if the cover is to be formfitted, close fitted, optionally reused. Resiliency is an elastomeric property which may be quantified by the tensile set value. The tensile set is defined as the relative permanent elongation of a sample, which remains after the sample has been stretched. Thus, a tensile set value of 0% corresponds to complete elastic recovery (i.e. no permanent elongation), and a tensile set value of 100% corresponds to zero elastic recovery.

For the cover to be functional it must further be shaped and fabricated to be impermeable to gasses and liquids. Depending on the device application, further requirements must be met. For example for a cover for an ultrasound device, it is essential that the cover is sufficiently ultrasound conductive, such that the cover itself is not attenuating the ultrasound pressure to a large degree.

A cover may have any shape, however a regular shape such as a tubular sheath with a closed end, is simple and cost-efficient to produce. A sheath with a closed end may for example be fabricated by welding a foil, or dip molding.

A polymeric cover may be made by dip molding by immersing a form, or mold, into a polymeric precursor solution, whereby the form or mold is coated by a film of precursor. The polymeric film may then be cured by e.g. heat treatment. Thus, dip molding can produce seamless covers, where the need of welding, as well as the risk of pinholes along the welded seam, may be avoided.

Covers have traditionally been made of natural rubber, such as latex. However, latex has the major disadvantage of being an allergenic material.

Polyurethane and polyisoprene have been suggested as an alternative material to covers of latex, since they may be used without causing the user or patient to suffer allergic reactions. However, covers of the non-allergenic or hypoallergenic materials with mechanical properties similar to latex, and which may be produced and shaped with the same efficiency and quality as latex, are difficult to produce.

In U.S. Pat. No. 4,684,490 [1] a polyurethane condom was made by dip molding in a prepolymer, which was subsequently cured to the elastomer at elevated temperatures of about 130-175° C. Condoms with elongation at break values of 640%, tensile set values of 5-7%, and thickness variation of 3.3 mils, corresponding to 7.62 microns, were disclosed.

In U.S. Pat. No. 6,329,444 [2] a polyisoprene cover suitable for e.g. gloves, catheters or condoms was made by repeated dip molding in a polyisoprene organic solution, and subsequently curing at 180° C. Balloon shaped articles with elongation at break above 720% and tensile set values below 5% were obtained.

Despite the advances in non-allergenic or hypoallergenic covers, there is a need for such covers which are more simple to produce, and which can be produced free of pinholes, with a uniform thickness, and which have improved ultrasound conductivity.

### SUMMARY OF INVENTION

The present invention provides a flexible cover for an ultrasound device. The cover comprises polyurethane, and is made by dip molding using low-temperature curing. Thus, the cover is non-allergenic in addition to being more simple, cost-efficient and environmental friendly to produce. At the same time the cover may be manufactured with a reduced number of pinholes, such as being free of pinholes, and with a high degree of uniform thickness. In addition, the cover provides an improved ultrasound conductivity, which is particularly advantageous for ultrasound devices operating in the frequency range from 2-20 MHz. Advantageously, the cover according to the present disclosure is used as a cover for an ultrasound device. Further advantageously, the cover may be used as a cover for other applications, having similar requirements to allergy, quality control, and production methods, For example, the cover is advantageously used as a cover for hands, and in a preferred embodiment, the cover is shaped as a glove or a mitten, such as gloves for medical treatment.

A first aspect of the invention relates to a flexible cover for an ultrasound device, the cover comprising polyurethane, wherein the cover is shaped as a sheath having a closed end, said shape is made by dip molding, and wherein the ultrasound attenuation is at least below 60% of the attenuation of latex, more preferably below at least 55%, 50%, 45%, 40%, 35%, or 30% below the attenuation of latex.

A second aspect of the invention relates to method of producing an ultrasound device cover, comprising the steps of:
providing one or more form(s), which are optionally pre-heated,
dipping the form(s) into an aqueous solution comprising one or more coagulant agents, whereby coagulant agent is attached to the surface of the form(s),
dipping the coagulant treated form(s) into an aqueous polyurethane dispersion, whereby a liquid film of polyurethane is coating the surface of the form(s),
drying the coated form(s) at a temperature below 100° C., whereby a coating of polyurethane is formed,
detaching the polyurethane coating from the form,

whereby a cover with a closed end is obtained.

A third aspect of the invention relates to a kit of parts comprising the cover according to the first aspect of the invention, an ultrasound gel, and optionally a bite guard.

A fourth aspect of the invention relates to the use of the cover according to the first aspect of the invention for ultrasound imaging, preferably within the frequency range between 2 to 20 MHz, more preferably between 6 to 20 MHz, and most preferably between 8 to 20 MHz

### DESCRIPTION OF DRAWINGS

The invention will in the following be described in greater detail with reference to the accompanying drawings.

FIG. 1 shows the damping (in dB) as a function of the frequency (in MHz) for six different ultrasound covers, where the only difference between the covers is the cover material. The damping, or ultrasound attenuation, was measured for three different latex covers (Latex I shown with square symbols, Latex II shown with circle symbols, Latex III shown with triangle symbols, where the apex is at the top), where the three latex covers were produced by ProDipp Medical, and for three different covers that were embodiments of the invention (FlexSeCo I shown with triangle symbols with apex at bottom, FlexSeCo II shown with triangle symbols with apex to the left, FlexSeCo III shown with triangle symbols with apex to the right). The polyurethane covers of the invention is also denoted as “FlexSeCo” or “Flexseco” in the present description, where FlexSeCo is a registered trademark by the applicant.

FIG. 2 shows the load at break (in N) for an embodiment of the invention (FlexSeCo 9921). For comparison, the load at break for comparative covers are included, where the comparative covers are made of: TPU (thermoplastic polyurethane from Civco, 610-1014, TOE), polyisoprene (from Civco), PCU (polycarbonate polyurethane, welded TOE), and latex of three different types (Latex 9921, Prodipp Medical, January 2014; Latex 9921, Prodipp Medical, light exposed; Latex, premier guard, 12-1203, thermo).

FIG. 3 shows the elongation (in %) for an embodiment of the invention (FlexSeCo 9921). For comparison, the load at break for comparative covers are included, where the comparative covers are made of: TPU (thermoplastic polyurethane from Civco, 610-1014, TOE), polyisoprene (from Civco), PCU (polycarbonate polyurethane, welded TOE), and latex of three different types (Latex 9921, Prodipp Medical, January 2014; Latex 9921, Prodipp Medical, light exposed; Latex, premier guard, 12-1203, thermo).

### DETAILED DESCRIPTION OF THE INVENTION

Ultrasound devices apply are common in medical diagnostics, since ultrasound can be used for imaging, or sonography, of internal body structures such as tendons, muscles, joints, vessels, and organs. Ultrasound is defined as sound waves with frequencies above 20 kHz. Examples of frequencies used for ultrasound imaging ranges from 20 kHz to 2 MHz and 4 GHz. The ultrasound device is advantageously protected by a cover to facilitate cleaning of the device as well as reduce the risks of spreading contagious diseases and cross-contamination between patients.

The resolution of an ultrasonic image will depend on the applied frequency, the degree of contact between the device and the structure to be imaged, and the damping, or attenuation, of the signal.

Shorter wavelengths allow for resolution of smaller details. However, shorter wavelengths also cause the object under examination to be exposed to a higher power density. Thus, advantageously for ultrasound imaging on human beings, the ultrasound is applied at lower wavelengths.

In a preferred embodiment of the invention, the cover is used for ultrasound imaging, preferably within the frequency range between 2 to 20 MHz, more preferably between 6 to 20 MHz, and most preferably between 8 to 20 MHz.

The contact between the ultrasound device and the structure to be imaged is also decisive for the resolution. The presence of a poor ultrasound conductor in the path of the sound wave will result in a poorer resolution of the image.

For example, air is a poor ultrasound conductor. Thus, air bubbles trapped between the ultrasound probe and/or the ultrasound cover and the object to be examined will result in areas that are not imaged, also known as “black spots”.

To avoid the presence of air pockets blocking the ultrasound transmission, an ultrasound transmission gel is typically applied. The gel itself is a good ultrasound conductor, and the gel ensures a high degree of contact between the probe, and/or optionally the cover, and the object to be examined.

Thus, ultrasound device covers may be supplied as part of a kit of parts, where the parts may include the cover and a gel. For ultrasound devices related to oral examination or oral entry into the body part, the kit of parts may further include a bite guard or a mouth guard.

An embodiment of the invention comprises a kit of parts comprising the cover according to the invention, an ultrasound gel, and optionally a bite guard.

The cover will inherently also dampen, or attenuate, the ultrasound signal. The ultrasound dampening of a cover will primarily depend on the cover thickness and the material of the cover. Inherently, better ultrasound resolution will be obtained the thinner the cover, and the more ultrasound conductive, or the less attenuating, the cover.

Polyurethane (PUR)

The present invention relates to a flexible cover comprising polyurethane or polyurethanes. Polyurethane(s), also abbreviated PUR and PU, are polymers composed of organic units joined by urethane links, which have the structural formula: (—NH—(C═O)—O—). The polyurethanes are typically synthesized by reacting di- or polyisocyanate with a polyol.

Depending on the polyurethane synthesis process and shaping method, polyurethanes can have variable properties and be used in applications as diverse as condoms, gaskets, and durable elastomeric wheels. The toxicity degree of a polyurethane will also depend on the synthesis process.

The present invention relates to a flexible cover comprising PUR, where the cover is shaped as a sheath having a closed end, and where the shape is made by dip molding. The synthesis process and shape were seen to provide a cover with surprisingly high ultrasound conductivity, or surprisingly low damping or attenuation of ultrasound. In particular, the attenuation of the covers was observed to be low compared to conventional latex. Thus, the PUR cover of the present invention was observed to be surprisingly suitable for a cover for an ultrasound device, and providing surprisingly high resolution for ultrasound imaging, and particularly real-time ultrasound imaging.

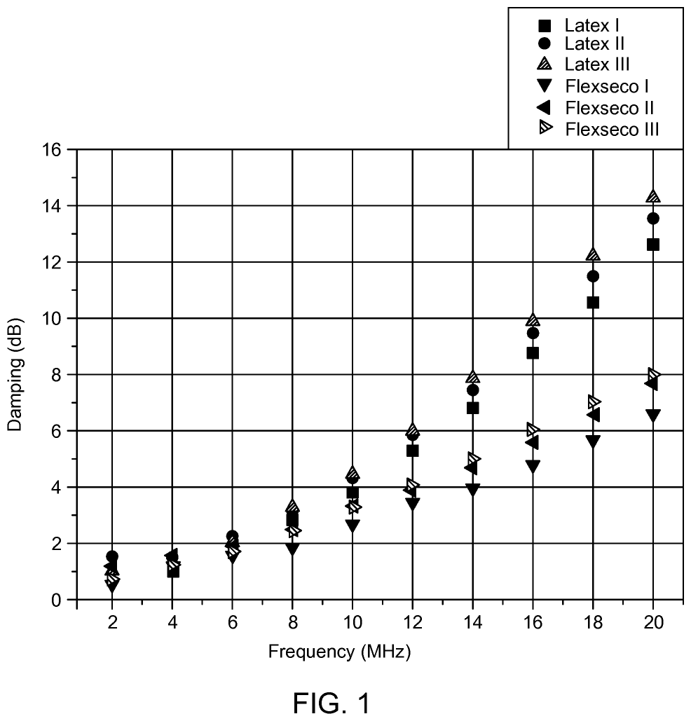

The ultrasound damping of covers according to the invention was tested as described in Example 2 and illustrated in FIG. 1. The damping, or ultrasound attenuation, was measured for three different covers according to the invention (FlexSeCo I shown with triangle symbols with apex at bottom, FlexSeCo II shown with triangle symbols with apex to the left, FlexSeCo III shown with triangle symbols with apex to the right). For comparison three different latex covers (Latex I shown with square symbols, Latex II shown with circle symbols, Latex III shown with triangle symbols, where the apex is at the top) were measured under identical and comparable conditions. From FIG. 1 it is seen that the covers of the present invention are configured to have a damping of at least 66% of the attenuation at frequencies between 12-20 MHz. For example at 20 MHz, the attenuation of latex is above 12 dB, and below 8 dB for PUR, corresponding to at least 67% lower damping for PUR. As another example, at 12 MHz the attenuation of latex is above 5 dB, and below 4 dB for PUR, corresponding to at least 80% lower damping for PUR.

An embodiment of the invention relates to a flexible cover for an ultrasound device, the cover comprising polyurethane, wherein the cover is shaped as a sheath having a closed end, said shape is made by dip molding, and wherein the ultrasound attenuation is at least below 65% or 60% of the attenuation of latex, more preferably below at least 55%, 50%, 45%, 40%, 35%, or 30% below the attenuation of latex.

In a further embodiment of the invention, the ultrasound attenuation of the cover is configured to be measured within the frequency range between 2 to 20 MHz, more preferably between 6 to 20 MHz, and most preferably between 8 to 20 MHz or 10 to 20 MHz.

In a further embodiment of the invention, the cover is configured such that the ultrasound attenuation of the cover is below 12 dB at 20 MHz, more preferably below 10 dB, and most preferably below 8 dB, and/or

wherein the ultrasound attenuation is below 10 dB at 18 MHz, more preferably below 8 dB, and most preferably below 7 dB, and/or

wherein the ultrasound attenuation is below 8 dB at 16 MHz, more preferably below 7 dB, and most preferably below 6 dB, and/or

wherein the ultrasound attenuation is below 6 dB at 14 MHz, more preferably below 5.5 dB, and most preferably below 5 dB, and/or

wherein the ultrasound attenuation is below 5 dB at 12 MHz, more preferably below 5.5 dB, and most preferably below 4 dB.

The cover of the present invention was further seen to have advantageous mechanical properties, making it further suitable as a cover for an ultrasound device. Thus, the mechanical properties facilitate that the covers may be configured to be formfitted, or close fitted, to a medical device, as well as being resilient to be reused. Thus, for example, the risk of trapped air bobbles are reduced.

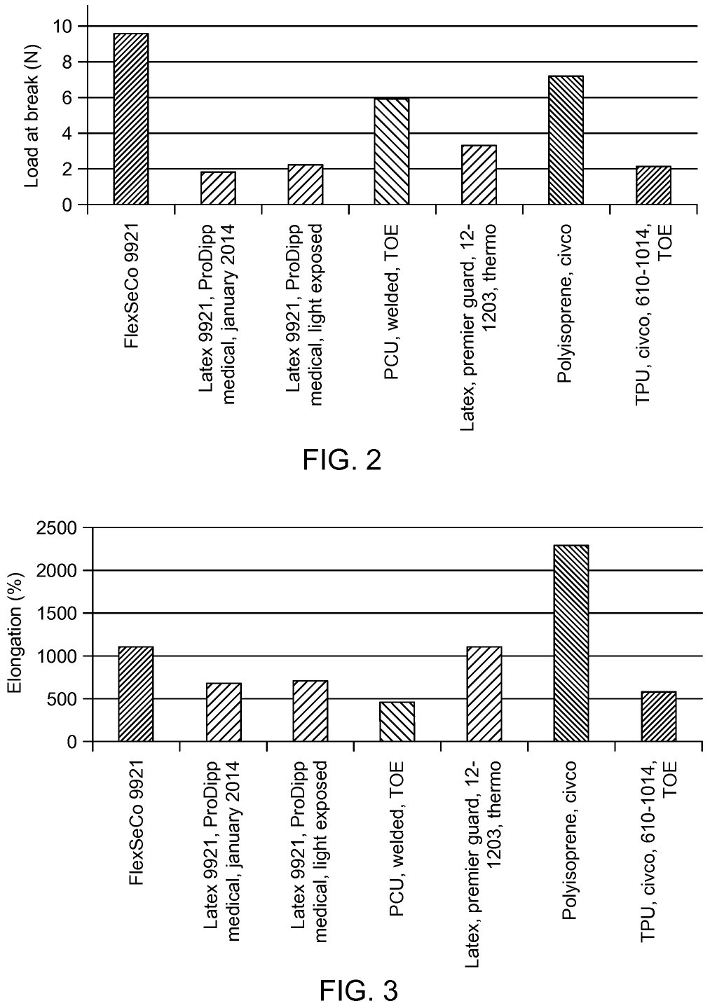

The mechanical properties of covers according to the invention was tested as described in Example 3 and illustrated in FIGS. 2 and 3. From the Figures it was seen that the PUR (FlexSeCo) covers have surprisingly high load at break compared to conventional and comparative latex samples, and further that the PUR covers have comparative or superior elongation properties compared to conventional and comparative latex samples.

The covers according to the invention was further found to have surprisingly low tensile set values. The “tensile set value” is the percent set after testing elongation, i.e. the deformation remaining immediately, or a defined period after stretching a sample. The mechanical tests were carried out as described in Example 3.

Advantageously, covers for an ultrasound device will have a low tensile set value and a high elongation at break and load at break, such that the be formfitted to a device.

In an embodiment of the invention, the cover is configured such that the tensile set is equal to or below ca. 6%, more preferably equal to or below 5, 4, 3, 2, or 1%.

In an embodiment of the invention, the cover is configured to having an elongation at break between 600-1000%, more preferably between 700-800%.

The covers according to the invention was further found to be non-allergenic or hypoallergenic, biocompatible, and environmental friendly to dispose, and/or simple to sterilize by e.g. ethylene oxide (EtO) sterilization, gamma- or e-beam sterilization. Thus, the covers are advantageously used for biological medical devices, such as being suitable as cover for an ultrasound device. Advantageously, the covers are made of polyurethane that is of a type qualified according to ISO 10993.

In an embodiment of the invention, the cover material is of medical grade. In a further embodiment of the invention, the cover material is ISO 10993 approved.

Shape

The dip molding, as described in the following section, includes that the covers of the present invention are made by a simple shaping and fabrication process, which further facilitates that the covers may be made seamless, impermeable to gasses and liquids, with a uniform wall, or film, thickness, and with variable shapes and sizes.

Advantageously, the covers are seamless such that the risks of pinholes, and gas or liquid permeation through the cover are reduced. Further, a seamless cover has the advantage of minimizing blocking and uneven disturbance of an ultrasound transmission.

In an embodiment of the invention, the cover is seamless.

The thinner the cover, the lower the ultrasound damping, and the better the potential ultrasound resolution. Furthermore, the more uniform the cover thickness, or the thickness of the film, or wall, of the cover, the better the resolution, since the risk of blocking and uneven disturbances of the ultrasound transmission is reduced.

In an embodiment of the invention, the thickness of the cover is between 20-220 microns, more preferably between 70-200 microns, and most preferably between 170-200 microns.

In a further embodiment, the thickness variation of the cover is equal to or below 30 microns, more preferably equal to or below 20 microns, and most preferably below 10 or 7 microns.

The dip molding facilitates that the covers of the present invention may be easily fabricated in variable shapes and sizes, since the shape and sizes are primarily determined by the initial form for dipping.

Advantageously, the covers are shaped such that they may be easily mounted or applied onto a device, e.g. by pulling over or rolling over in a similar manner as a glove or a condom. Thus, advantageously the cover is shaped as a sheath having a closed end. For simple fabrication and simple fabrication of the forms or molds, it is further advantageous that the sheath has a regular geometric shape, such as a tube with a closed end, or a cone shaped tube, or a tube with a flared, or tapered, entry portion.

In an embodiment of the invention, the cover is shaped as a tube with a closed end. In a further embodiment, the cover is a cone shaped tube. In a further embodiment, the cover is a tube with a flared, or tapered, entry portion.

To facilitate the mounting or application of a cover, or to facilitate the removal of the article from the form after drying and curing, it may be advantageous the cover comprises a lubricant, or a material with a low coefficient of friction, such as talc or talcum. However, to minimize the risk of the cover being slippy and difficult to handle, it is advantageous that the amount of lubricant is low.

In an embodiment of the invention, the cover comprises talc. In a further embodiment, the cover comprises below 10 wt % talc, more preferably below 8, 6, 4, 2 wt % talc, and most preferably below 1 wt % talc.

Ultrasound devices may have any shapes and sizes, however typical devices includes a longitudinal extending element, such as an elongated arm or probe. Thus, advantageously, the cover is adapted to elongated shapes.

In an embodiment of the invention, the cover is a tube wherein the diameter of the tube is between 0.1-10 cm, more preferably between 0.5-5 cm, and most preferably between 0.6-3 cm.

In a further embodiment of the invention, the length of the cover is above 30 cm, more preferably above 40, 50, 60, 70, 80, 90 cm, and most preferably above 100 cm.

By similar simple shaping and fabrication processes, covers of different shapes may be formed. For example, covers for hands, such as gloves or mittens, having at least one closed end, may be formed by dip molding. Thus, covers for hands, which are seamless, and has a low risk of pinholes, gas or liquid permeation, and further has a low thickness and a uniform thickness, such that it provides improved ultrasound transmission, may be formed. The manufacturing of a glove is further described in Example 4.

In an embodiment of the disclosure, the cover is used as a cover for hands and/or a glove.

Dip Molding

The covers of the present invention are made by dip molding. An example of a PUR cover made by dip molding is described in Example 1.

As described in Example 1 dip molding involves dipping a form, whose outer surface has the configuration of the article to be formed, in a liquid medium that contains a liquefied polymer. Thus, if the article to be formed is a sheath having a closed end, e.g. a condom, the form may be a mandrel or a tube with a closed end.

The form may also be referred to as a mold. The form may be made or based on any material that is not reactive with the liquid medium, such as glass, polymers, metals and coated metals. Examples of materials include stainless steel, galvanized steel or iron, iron (Fe), aluminium (Al), zink (Zn), carbides, or any combinations thereof.

Upon withdrawal of the form from the liquid, a film of the liquid will cover the surface of the form, thus forming a coating. The properties of the liquid film will depend on the type of liquefied polymer as well as process parameters such as liquid temperature, pH, duration of dipping, and how the form is dipped into the liquid.

The liquid film still placed on the form is subsequently dried and the polymer cured. The dried and cured film will then have the final shape of the article, and the article is finally removed from the form.

A series of dipping, drying, and optionally curing, cycles may be needed to build up film thickness. Alternatively, a separate dipping step in a coagulant solution may be used to help build up film thickness. The form is then dipped in the coagulant solution before dipping of the first polymer layer. The coagulant is typically a solution comprising calcium salt(s), which facilitates the formation of the liquid film, and thus thicker wall thickness of the final article.

By the term curing is meant toughening or hardening of the polymer material by cross-linking of the polymer chains. Curing may be activated by heat, electron beam, or chemicals additives, which again may be activated by ultraviolet radiation. The specific process of curing of natural rubber is also referred to as vulcanization.

The degree of curing will depend on the curing process and the type of liquefied polymer. The polymer is typically in the form of an emulsion, or a solution of the polymer, or a pre-polymer, in an organic solvent. For example liquefied latex is typically an aqueous emulsion, where the polymer is the dispersed phase and water or an aqueous solution is the continuous phase. The curing step is typically activated by heat, and carried out at at elevated temperatures far above 100° C.

The polyurethane cover of the current invention is based on an aqueous dispersion of solid polyurethane particles, i.e. the polyurethane is synthesized. The particles are advantageously made of polyurethanes tested and qualified according to ISO 10993.

An embodiment of the invention includes a method of producing an ultrasound device cover, comprising the steps of:
providing one or more form(s), which are optionally pre-heated,
dipping the form(s) into an aqueous solution comprising one or more coagulant agents, whereby coagulant agent is attached to the surface of the form(s),
dipping the coagulant treated form(s) into an aqueous polyurethane dispersion, whereby a liquid film of polyurethane is coating the surface of the form(s),
drying the coated form(s) at a temperature below 100° C., whereby a coating of polyurethane is formed,
detaching the polyurethane coating from the form,

whereby a cover with a closed end is obtained.

Aqueous Dispersion

The aqueous dispersion according to the present invention comprises the polymer in the form of solid particles dispersed in a continuous aqueous solution. This is in contrast to a liquefied polymer emulsion or organic solution, where the polymer phase also is in the liquid state.

The solid load content of the aqueous dispersion affects the wall thickness variation of the article to be formed, and the degree of control of the wall thickness variation. The lower the solid load, the lower the thickness variation. Furthermore, dip molding is inherently associated with variations in the wall thickness. When a form is immersed and subsequently withdrawn out of the liquid medium, the bottom part of the form will have had longer contact time with the liquid, and the film thickness will therefore be thicker in the bottom part.

Advantageously, the polymer dispersion has a low solid load content, i.e. the load of polymer is below 70 wt %, more preferably below 60, 50, or 40 wt %. In an embodiment of the invention, the solid load of the aqueous dispersion is below 70 wt %, more preferably below 60, 50, 40 wt %.

The use of the aqueous dispersion with a low solid load content further facilitates that the dip molding process more efficient as well as more environmental friendly. Thus, the use of an organic solvent is avoided, where an organic solvent typically implies concerns with regard to work safety as well as disposal of the solvent after use. Furthermore, more environmental friendly and biological compatible solids may be used, such as ISO 10993 approved PUR.

In an embodiment of the invention, the dip molding is carried out in an aqueous dispersion. In a further embodiment of the invention, the aqueous dispersion comprises particles of polyurethane that is ISO 10993 approved.

The aqueous dispersion may further facilitate that the curing step may be sufficiently carried out at temperatures below 100° C. The lower temperatures further result in a faster and more cost-efficient process.

In an embodiment of the invention, the dip molding includes curing at a temperature below 100° C., more preferably below 90, 80, 70° C.

The aqueous dispersion further facilitates the use of further additives, which may facilitate the removal of the article from the form after drying and curing. Further, the further additives may improve the wetting properties of the aqueous dispersion, and thus reduce the thickness variations of the cover. The thickness variation is affected by the properties of the liquid medium, such as the wetting properties. Furthermore, an aqueous dispersion will have wetting properties different from an emulsion or solution, and the use of an aqueous dispersion therefore enables reduced thickness variation.

Further advantageously, an aqueous dispersion may include one or more anti-bacterial agent(s), such as silver particles or precursors. Thus, the manufactured product may obtain anti-bacterial properties.

The further additives may further improve the film formation, such as coagulation agent(s).

In an embodiment of the invention, the dip molding includes one or more coagulation agent(s), and/or one or more wetting agent(s), and/or one or more anti-bacterial agent(s).

### EXAMPLES

The invention is further described by the examples provided below.

### Example 1: Polyurethane Cover Formed by Dip Molding

Forms with the shape of a mandrel were used. The mandrels are optionally cleaned and pre-heated before the dipping process.

An aqueous dispersion of pre-synthesized polyurethane was prepared. The synthesized polyurethane may be any type of polyurethane that is approved according to ISO 10993. The dispersion was based on water, and the solid load of polyurethane was low, ca. 40 wt %, such that the viscosity of the dispersion was low, and the pH between 3-8.

The mandrel was first treated with a coagulant following the conventional procedures well known from latex dip molding.

The coagulant treated mandrel was then dipped in the aqueous polyurethane dispersion for a time sufficient to form a liquid film thickness between 175-195 μm.

The coated mandrel was dried in a furnace at a temperature below 100° C., such as a temperature of ca. 75° C. or 75° C.±10° C. The liquid film around the mandrel was thereby cured to form the final cover.

The cured cover was removed from the mandrel. Optionally the cover is cleaned with ethanol, and/or treated with talc to improve the handling of the covers.

The process results in a polyurethane cover of FlexSeCo, such as FlexSeCo 9921.

### Example 2: Ultrasound Testing

Polyurethane covers were manufactured using the method described in Example 1. Three different covers from three different batches of aqueous dispersions were manufactured, and the damping, or ultrasound attenuation, was measured between frequencies of 2 to 20 MHz.

The testing conditions were configured to be identical as known to the skilled person, such that the measurements were comparable. For comparison, the damping of three different latex covers were also tested under identical conditions.

FIG. 1 shows the measured damping (in dB) as a function of the frequency (in MHz) for the six different ultrasound covers, where the only difference between the covers is the cover material. The damping, or ultrasound attenuation, was measured for three different latex covers (Latex I shown with square symbols, Latex II shown with circle symbols, Latex III shown with triangle symbols, where the apex is at the top), and for three different covers that were embodiments of the invention (Flexseco I shown with triangle symbols with apex at bottom, Flexseco II shown with triangle symbols with apex to the left, Flexseco III shown with triangle symbols with apex to the right). The three latex covers were produced by ProDipp Medical.

At the measured frequencies between 10 to 20 MHz, the ultrasound damping, or attenuation, is clearly seen to be lower for the polyurethane covers according to the invention compared to the latex covers. For example, at 20 MHz the attenuation is reduced from 12-14 dB for latex, to 6-8 dB for the polyurethane covers, and at 12 MHz the attenuation is reduced from 5-6 dB for latex, to 3-4 dB for the polyurethane covers

(FlexSeCo). In general, the relative reduction in the attenuation for the polyurethane corresponds to an attenuation 66% below the attenuation of latex.

The reduced attenuation of the ultrasound covers of polyurethane of the present invention facilitates improved resolution in ultrasound imaging. The improved resolution may be seen by comparative ultrasound imaging, where the imaging is performed with respectively a conventional latex cover, and a polyurethane cover according to the present invention. Particularly, improved real-time imaging may be obtained with the ultrasound covers according to the invention.

### Example 3: Mechanical Testing

Polyurethane covers were manufactured using the method described in Example 1, and subjected to mechanical testing. The manufactured covers were from the same batch (Flexseco 9921), and for statistical reasons ca. ten samples were prepared and tested.

The testing was carried out in accordance with the General Inspection Level I, ISO 2859-1, and the tensile testing was further carried out to be predominantly in compliance with DS/EN 455-2.

The samples were sampled from the thinner part of the cover, i.e. the part placed at the upper end of the form. The samples were cut to be 3±0.5 mm wide, and the samples were placed in a uniaxial tensile testing device configured to a sample test length of 2±2 cm.

The uniaxial testing device was equipped with a load cell of 500 N, and set to a crosshead speed of 500 mm/minute, and with break detector settings of 0.25 N and 30%.

For comparison, covers made of different materials were tested under identical conditions. Ca. ten samples of each material were tested, and the comparative covers include: TPU (thermoplastic polyurethane from Civco, 610-1014, TOE), polyisoprene (from Civco), PCU (polycarbonate polyurethane, welded TOE), and latex of three different types (Latex 9921, Prodipp, January 2014; Latex 9921, Prodipp, light exposed; Latex, premier guard, 12-1203, thermo).

The results from the mechanical tests are summarised in FIGS. 2, 3, and Table 1.

FIG. 2 shows the load at break (in N) for the embodiment of the invention (FlexSeCo 9921), and the comparative covers (TPU (thermoplastic polyurethane from Civco, 610-1014, TOE), polyisoprene (from Civco), PCU (polycarbonate polyurethane, welded TOE), and latex of three different types (Latex 9921, Prodipp Medical, January 2014; Latex 9921, Prodipp Medical, light exposed; Latex, premier guard, 12-1203, thermo)).

FIG. 3 shows the elongation (in %) for the embodiment of the invention (FlexSeCo 9921), and the comparative covers are included (TPU (thermoplastic polyurethane from Civco, 610-1014, TOE), polyisoprene (from Civco), PCU (polycarbonate polyurethane, welded TOE), and latex of three different types (Latex 9921, Prodipp Medical, January 2014; Latex 9921, Prodipp Medical, light exposed; Latex, premier guard, 12-1203, thermo)).

FIG. 2 shows that a significant higher load at break was observed for the PUR cover (FlexSeCo 9921) according to the embodiment of the invention. The load of break was above 9 N, whereas the comparative covers showed load of break between ca. 2-7 N. In particular it was observed that the PUR samples according to the invention were superior in strength to latex, which showed a ca. 3 times lower load of break (i.e. between 2-3 N).

FIG. 3 shows that the PUR cover (FlexSeCo 9921) according to the embodiment of the invention, has comparative or superior elongation properties, compared to latex. An elongation of ca. 1100% was observed for the PUR cover, whereas the latex samples showed elongation between ca. 600-1100%.

TABLE 1
Summary of the mechanical tests and the results.

| | | Number of | | | | |
|---|---|---|---|---|---|---|
| No. | Sample information | samples tested | Median | Max | Min | Test comment |
| 1 | PUR-1 | 10 | 9.6 | 10.7 | 6.0 | Load at break (N) |
| | (FlexSeCo 9921) | | | | | |
| 2 | PUR-1 | 10 | 1111 | 1167 | 1014 | Elongation (%) |
| | (FlexSeCo 9921) | | | | | |
| 3 | Latex-1 | 10 | 1.8 | 3.8 | 1.5 | Load at break (N), one |
| | (Latex 9921, januar 2014, | | | | | sample did not break |
| | ProDipp Medical). | | | | | |
| 4 | Latex-1 | 10 | 687 | 786 | 619 | Elongation (%), one |
| | (Latex 9921, januar 2014, | | | | | sample did not break |
| | ProDipp Medical) | | | | | (730%) |
| 5 | Latex-2 | 10 | 2.2 | 3.2 | 1.1 | Load at break (N), two |
| | (Latex 9921, ProDipp | | | | | samples did not break |
| | Medical, light exposed) | | | | | (1.3 N og 1.1 N) |
| 6 | Latex-2 | 10 | 715 | 857 | 661 | Elongation (%), two |
| | (Latex 9921, ProDipp | | | | | samples did not break |
| | Medical, light exposed) | | | | | (both 638%) |
| 7 | Latex | 10 | 454 | 473 | 431 | Elongation (%), all |
| | (PCU, welded, TOE) | | | | | breaks within the |
| | | | | | | welding |
| 8 | Latex-3 | 9 | 3.3 | 6.6 | 2.7 | Load at break (N) |
| | (Latex, premier Guard, 12- | | | | | |
| | 1203, Thermo Transducer | | | | | |
| | cover) | | | | | |
| 9 | Latex-3 | 9 | 1115 | 1802 | 651 | Elongation (%) |
| | (Latex, premier Guard, 12- | | | | | |
| | 1203, Thermo Transducer | | | | | |
| | cover) | | | | | |
| 10 | Polyisoprene-1 | 8 | 7.2 | 9.9 | 2.4 | Load at break (N) |
| | (Polyisoprene, Civco) | | | | | |
| 11 | Polyisoprene-1 | 8 | 2295 | 2369 | 1463 | Elongation (%) |
| | (Polyisoprene, Civco) | | | | | |
| 12 | TPU-1 | 10 | 2.1 | 2.4 | 1.5 | Load at break (N) |
| | (TPU, Civco, 610-1014, | | | | | |
| | TOE) | | | | | |
| 13 | TPU-1 | 10 | 548 | 582 | 451 | Elongation (%) |
| | (TPU, Civco, 610-1014, | | | | | |
| | TOE) | | | | | |

### Example 4: Polyurethane Cover Formed by Dip Molding and Suitable as a Glove

Polyurethane covers were manufactured using the method described in Example 1, using a form adapted for shaping a glove. Thus, polyurethane covers suitable as a glove were made. The covers are subjected to mechanical testing, and damping, or ultrasound attenuation, is measured between frequencies of 2 to 20 MHz.

### REFERENCES

[1] U.S. Pat. No. 4,684,490
[2] U.S. Pat. No. 6,329,444

1. A flexible cover for an ultrasound device, the cover comprising polyurethane, wherein the cover is shaped as a sheath having a closed end, said shape is made by dip molding, and wherein the ultrasound attenuation is at least below 65% or 60% of the attenuation of latex.

2. The cover according to claim 1, wherein the ultrasound attenuation of the cover is below 12 dB at 20 MHz, or
wherein the ultrasound attenuation is below 10 dB at 18 MHz, or
wherein the ultrasound attenuation is below 8 dB at 16 MHz, or
wherein the ultrasound attenuation is below 6 dB at 14 MHz, or
wherein the ultrasound attenuation is below 5 dB at 12 MHz.

3. The cover according to claim 1, wherein the tensile set is equal to or below ca. 6%.

4. The cover according to claim 1, wherein the cover is seamless.

5. The cover according to claim 1 further comprising talc.

6. The cover according to claim 5 comprising below 10 wt % talc.

7. The cover according to claim 1, wherein the cover material is ISO 10993 approved.

8. The cover according to claim 1, wherein the dip molding includes one or more coagulation agent(s).

9. The cover according to claim 1, wherein the dip molding includes curing at a temperature below 100° C.

10. The cover according to claim 1, wherein the dip molding is carried out in an aqueous dispersion.

11. (canceled)

12. The cover according to claim 10, wherein the solid load of the aqueous dispersion is below 70 wt %.

13. The cover according to claim 1 having an elongation at break between 600-1000%.

14. The cover according to claim 1 having a thickness between 20-220 microns.

15. The cover according to claim 1, wherein the thickness variation is equal to or below 30 microns.

16. The cover according to claim 1 shaped as a tube with a closed end.

17. The cover according to claim 16, wherein the tube is a cone shaped tube.

18. The cover according to claim 16, wherein the diameter of the tube is between 0.1-10 cm.

19. The cover according to claim 1, wherein the length of the cover is above 30 cm.

20. A method of producing an ultrasound device cover, comprising the steps of:
providing one or more form(s),
dipping the form(s) into an aqueous solution comprising one or more coagulant agents, whereby coagulant agent is attached to the surface of the form(s),
dipping the coagulant treated form(s) into an aqueous polyurethane dispersion, whereby a liquid film of polyurethane is coating the surface of the form(s),
drying the coated form(s) at a temperature below 100° C., whereby a coating of polyurethane is formed,
detaching the polyurethane coating from the form,
whereby a cover with a closed end is obtained.

21. (canceled)

22. (canceled)

23. A kit of parts comprising the cover according to claim 1, an ultrasound gel, and optionally a bite guard.

24. (canceled)
